(19) United States
(12) Patent Application Publication
(10) Pub. No.: US 2026/0148083 A1
(43) Pub. Date: May 28, 2026

(54) SELECTIVE ACQUISITION FOR MULTI-MODAL TEMPORAL DATA

(71) Applicant: DeepMind Technologies Limited, London (GB)

(72) Inventors: Jannik Lukas Kossen, Nordhorn (DE); Danielle Charlotte Mary Belgrave, London (GB); Nenad Tomasev, London (GB); Catalina-Codruta Cangea, Cambridge (GB); Sofia Ira Ktena, London (GB); Eszter Vértes, London (GB); Viorica Patraucean, London (GB); Andrew Coulter Jaegle, New York, NY (US)

(21) Appl. No.: 19/122,063

(22) PCT Filed: Oct. 21, 2023
(86) PCT No.: PCT/EP2023/079389

§ 371 Date: Apr. 17, 2025

(30) Foreign Application Priority Data
Oct. 21, 2022 (GR) 20220100868

(51) Int. Cl. G06N 3/092; G16H 50/20

(52) U.S. Cl. CPC G06N 3/092; G16H 50/20

(57) ABSTRACT

Methods, systems, and apparatus, including computer programs encoded on computer storage media, for generating a prediction characterizing an environment. In one aspect, a method includes obtaining a respective observation characterizing a state of an environment for each time step in a sequence of multiple time steps, comprising, for each time step after a first time step in the sequence of time steps: processing a network input that comprises observations obtained for one or mor preceding time steps to generate a plurality of acquisition decisions; obtaining an observation for the time step, wherein the observation includes data corresponding to modalities that are selected for acquisition at the time step, does not include data corresponding to modalities that are not selected for acquisition at the time step; and processing a model input that includes the observation for each time step in the sequence of time steps to generate the prediction.

### CROSS-REFERENCE TO RELATED APPLICATIONS

This application claims priority to GR national application No. 20220100868, filed on Oct. 21, 2022. The disclosure of the prior application is considered part of and is incorporated by reference in the disclosure of this application.

### BACKGROUND

This specification relates to processing data using machine learning models.

Machine learning models receive an input and generate an output, e.g., a predicted output, based on the received input. Some machine learning models are parametric models and generate the output based on the received input and on values of the parameters of the model.

Some machine learning models are deep models that employ multiple layers of models to generate an output for a received input. For example, a deep neural network is a deep machine learning model that includes an output layer and one or more hidden layers that each apply a non-linear transformation to a received input to generate an output.

### SUMMARY

This specification generally describes a system implemented as computer programs on one or more computers in one or more locations that generates a prediction characterizing an environment.

According to one aspect, there is provided a method performed by one or more computers, the method comprising: obtaining a respective observation characterizing a state of an environment for each time step in a sequence of multiple time steps, comprising, for each time step starting from a first time step in the sequence of time steps: processing a network input that comprises observations obtained for any preceding time steps using a selection neural network to generate a plurality of acquisition decisions, wherein each acquisition decision corresponds to a respective modality from a set of multiple modalities and defines whether data corresponding to the modality is selected for acquisition at the time step; obtaining an observation for the time step, wherein the observation includes only data corresponding to modalities selected for acquisition at the time step; and processing a model input that includes the observation for each time step in the sequence of time steps using a prediction model to generate a prediction characterizing the environment.

In some implementations, the method further comprises: determining an acquisition cost based on the respective modalities selected for acquisition at each time step in the sequence of time steps; determining a reward based at least in part on the acquisition cost; and training the selection neural network based on the reward using a reinforcement learning technique.

In some implementations, each modality in the set of modalities is associated with a respective cost factor, and wherein determining the acquisition cost comprises: determining, for each time step in the sequence of time steps, a respective acquisition cost for the time step based on the respective cost factor associated with each modality selected for acquisition at the time step; and determining the acquisition cost as a combination of the acquisition costs for the time steps.

In some implementations, for each time step in the sequence of time steps, determining the acquisition cost for the time step comprises: determining the acquisition cost for the time step as a sum of the cost factor associated with each modality selected for acquisition at the time step.

In some implementations, determining the acquisition cost as a combination of the acquisition costs for the time steps comprises: determining the acquisition costs as a sum over the acquisition costs for the time steps.

In some implementations, for one or more of the modalities, the cost factor for the modality is based at least in part on an amount of resource usage required to capture data corresponding to the modality.

In some implementations, the resource usage required to capture data corresponding to the modality characterizes at least energy usage required to capture data corresponding to the modality.

In some implementations, the resource usage required to capture data corresponding to the modality characterizes at least an amount of time required to capture data corresponding to the modality.

In some implementations, for one or more of the modalities, the cost factor for the modality is based at least in part on a risk associated with capturing data corresponding to the modality.

In some implementations, the environment comprises a patient, and the risk associated with capturing data corresponding to the modality is based at least in part on a medical risk to the patient resulting from capturing data corresponding to the modality.

In some implementations, the method further comprises: determining a prediction error that measures an error in the prediction generated by the prediction model; and determining the reward based on both: (i) the acquisition cost, and (ii) the prediction error.

In some implementations, the prediction model is a machine learning model.

In some implementations, the prediction model comprises a neural network.

In some implementations, the method further comprises: training the prediction machine learning model to optimize an objective function that depends on a prediction error of the prediction machine learning model.

In some implementations, for each time step in the sequence of time steps, the network input to the selection neural network at the time step further comprises: data identifying the acquisition decision for any modality at any preceding time step.

In some implementations, the method further comprises: for each of one or more time steps in the sequence of time steps: processing a model input that includes the observation for the time step and observations for one or more preceding time steps in the sequence of time steps using the prediction model to generate an intermediate prediction characterizing the environment; and determining an intermediate prediction error that measures an error in the intermediate prediction generated by the prediction model; and determining the reward based at least in part on the intermediate prediction errors.

In some implementations, the set of modalities includes an imaging modality, and wherein data corresponding to the imaging modality comprises image data.

In some implementations, the set of modalities includes a medical imaging modality.

In some implementations, the environment is a medical environment that comprises a patient.

In some implementations, the prediction characterizing the environment comprises a predicted medical diagnosis of the patient.

In some implementations, the prediction characterizing the environment comprises a prediction for a medical treatment to be applied to the patient.

In some implementations, the method further comprises, for each time step after the first time step in the sequence of time steps: determining that: (i) data corresponding to modalities, from the set of modalities, that are selected for acquisition at the time step will be included in the observation for the time step, and (ii) data corresponding to modalities, from the set of modalities, that are not selected for acquisition at the time step will not be included in the observation for the time step.

In some implementations, for each of one or more time steps after the first time step in the sequence of time steps: only a proper subset of the modalities in the set of modalities are selected for acquisition at the time step.

In some implementations, the method further comprises, for each time step after the first time step in the sequence of time steps: causing data to be acquired only for modalities selected for acquisition at the time step.

According to another aspect there is provided a system comprising: one or more computers; and one or more storage devices communicatively coupled to the one or more computers, wherein the one or more storage devices store instructions that, when executed by the one or more computers, cause the one or more computers to perform the operations of the methods described herein.

According to another aspect there are provided one or more non-transitory computer storage media storing instructions that when executed by one or more computers cause the one or more computers to perform the operations of the methods described herein.

The subject matter described in this specification can be implemented in particular implementations so as to realize one or more of the following advantages.

This specification describes a system for processing multi-modal data captured over a sequence of time points to generate a prediction characterizing an environment. In many real-world scenarios, capturing data corresponding to a modality can incur significant cost, e.g., in terms of resource consumption (e.g., consumption of energy or time), or in terms of risk (e.g., medical risk, e.g., resulting from exposing a patient to radiation from acquiring medical images of the patient, e.g., x-ray images or CT images). Moreover, processing data corresponding to certain modalities can also incur significant cost, e.g., in terms of computational resources (e.g., memory and computing power), e.g., for high-dimensional data such as image data, video data, or audio data. The system described in this specification can adaptively determine which data modalities to acquire at each time point, and for certain time points, can acquire fewer than all the available modalities (or can even refrain from acquiring any modalities).

The system can be trained, using machine learning techniques, to optimize a trade-off between acquisition cost and predictive performance. In particular, the system can be trained to achieve an acceptable predictive performance while minimizing acquisition cost across the available modalities, thus enabling more efficient use of resources (e.g., energy resources or computational resources) and reduction of risk (e.g., medical risk). In some cases, the system can be trained to optimize the predictive performance while encouraging (or requiring) acquisition costs to satisfy a cost budget.

The details of one or more implementations of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example neural network system.

FIG. 2 is a flow chart of an example process for generating a prediction characterizing an environment.

FIG. 3 is a flow chart of an example process for training a selection neural network.

FIG. 4 is a flow chart of an example process for sub-steps of one of the steps of the process of FIG. 3.

FIG. 5 is a flow chart of another example process for sub-steps of one of the steps of the process of FIG. 3.

FIG. 6 is an example illustration of generating a prediction using the selection neural network and determining one or more updates to the parameter values of the selection neural network based on the prediction.

Like reference numbers and designations in the various drawings indicate like elements.

### DETAILED DESCRIPTION

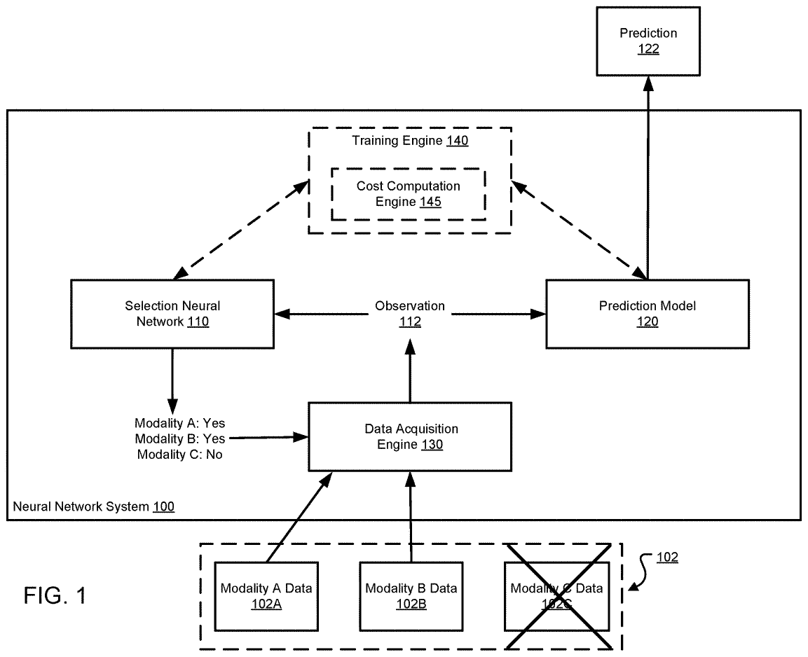

FIG. 1 shows an example neural network system 100. The neural network system 100 is an example of a system implemented as computer programs on one or more computers in one or more locations, in which the systems, components, and techniques described below can be implemented.

The neural network system 100 includes a selection neural network 110, a prediction model 120, a data acquisition engine 130, and, optionally, in some implementations, a training engine 140.

Generally, the neural network system 100 is a system that acquires input data 102 that is generated within or about an environment at each time step over a sequence of multiple time steps, and makes one or more predictions 122 that characterize one or more aspects of the environment. For example, the neural network system 100 can output a prediction 122 after having acquired input data 102 for the last time step in the sequence of multiple time steps.

At any given time step in the sequence, the input data 102 that is acquired by the system can potentially (but not necessarily) include data from a set of two or more available modalities. In this specification, a data “modality” refers to a type of data, e.g., that is generated using a specified sensor or diagnostic technique (e.g., medical diagnostic technique).

The set of modalities can include any appropriate modalities. A few examples of possible modalities are described next. In some implementations, the set of modalities comprises one or more of these examples.

In some implementations, the set of modalities includes an imaging modality, and data corresponding to the imaging modality comprises image data (e.g., one-dimensional (1D) image data, two-dimensional (2D image data, three-dimensional (3D) image data, etc.). The image data may comprise pixel value data, e.g. color or monochrome pixel value data.

For instance, the set of modalities can include one or more medical imaging modalities, e.g., a computed tomography (CT) modality, an ultrasound (US) modality, a magnetic resonance imaging (MRI) modality, an x-ray modality, a histological imaging modality, an electroencephalogram (EEG) modality, an electromyography (EMG) modality, an electrocardiogram (ECG) modality, etc.

As another example, the set of modalities can include a camera modality, e.g., where data corresponding to the camera modality is captured using a camera, e.g., a visible spectrum camera or an infrared spectrum camera.

In some implementations, the set of modalities can include a genetic data modality, and data corresponding to the genetic data modality comprises genetic data. Genetic data can include, e.g., data defining a respective expression level (in a subject) of each gene in a set of genes. The genetic data may be obtained by a suitable diagnostic technique, such as DNA or RNA sequencing, performed on genetic material obtained from the subject.

In some implementations, the set of modalities can include a proteomic data modality, and data corresponding to the proteomic data modality comprises proteomic data. Proteomic data can include, e.g., data defining a respective expression level (in a subject) of each protein in a set of proteins.

In some implementations, the set of modalities can include a blood testing modality, and data corresponding to the blood testing modality can include data defining levels of one or more components of the blood of a subject, e.g., sodium, potassium, chloride, bicarbonate, blood urea nitrogen, magnesium, creatinine, glucose, calcium, cholesterol, etc.

In some implementations, the set of modalities can include an audio modality, and data corresponding to the audio modality can include audio data, e.g., audio data characterizing words spoken by a person, audio data characterizing sounds made by one or more body parts of a person (e.g., the heart, the digestive system, the lungs, etc.), etc. The audio data may comprise data defining an audio waveform such as a series of values in the time and/or frequency domain defining the waveform.

In some implementations, the set of modalities can include a biopsy modality, and data corresponding to the biopsy modality can characterize a sample of cells or tissue obtained from a patient by way of a biopsy. For instance, data corresponding to the biopsy modality can include a microscope image of the sample obtained from the patient.

In some implementations, the set of modalities can include modalities that measure one or more of: humidity, light, air quality, sound, temperature, wind speed, pH, etc.

In a particular implementation, the set of modalities includes at least: a medical imaging modality and a blood testing modality.

In a particular implementation, the set of modalities includes at least: a medical imaging modality, a blood testing modality, and a biopsy modality.

In a particular implementation, the set of modalities includes at least: a medical imaging modality, a blood testing modality, a biopsy modality, and a genetic data modality.

In some of these implementations, these data types differ not only in feature spaces and dimensionalities, but also in data capturing processes and costs associated with capturing data corresponding to these data types. For example, medical imaging can be ordered at the discretion of a physician and needs to be captured at a relatively higher cost, e.g., in terms of resource consumption or in terms of risk, while blood pressure and temperature can be monitored on a regular basis and can be captured at a relatively lower cost.

The environment can be any appropriate environment, e.g., a real-world environment, e.g., a medical environment, an agriculture environment, an aquaculture environment, an industrial environment, or a scientific environment.

A medical environment can include a patient, and one or more of the modalities can be modalities that generate data characterizing the patient, as described above.

An industrial environment can include, e.g., a manufacturing facility (e.g., that includes one more industrial machines used for the production of manufactured goods), a chemical processing facility (e.g., that includes one more industrial machines used for chemical processing), a data center facility (e.g., that includes a collection of computing units, e.g., processors, used for performing computing tasks), or an energy production facility (e.g., a nuclear plant, a hydroelectric plant, a photovoltaic power station, etc.). The one or more modalities can be modalities that generate data characterizing the facility, e.g. data generated by one or more sensors located within or around the facility, e.g. sensors for measuring the states of industrial machines or computing units within the facility. A prediction characterizing the industrial environment may comprise predicted values for one or more properties that may be determined based on the sensor values, or one or more properties measured by the sensor(s), e.g. the predicted values may comprise predicted sensor values.

A scientific environment can include a collection of subjects being studied for scientific purposes, where the subjects can include, e.g., plants, animals, cells, tissues, etc.

The environment can evolve over time, and thus the input data 102 generated within or about the environment at a first time step can have different values than the input data 102 generated within or about the environment at a second time step. The collection of input data 102 that is acquired over the sequence of multiple time steps may thus be referred to as a “temporal” sequence input data, because in some implementations, the input data is arranged according to the time step at which it was captured. For example, the most recent input data is the last input data in a temporal sequence of input data and the least recent input data is the first input data in the temporal sequence.

The one or more predictions 122 that characterize one or more aspects of the environment are made by the prediction model 120 based on the input data 102. The prediction model 120 can be configured as a machine learning model that can have any appropriate machine learning model architecture. For instance, the prediction model 120 can be implemented as a neural network, or a decision tree, or a random forest, or a support vector machine, or a linear regression model, and so forth. In a particular example, the prediction model 120 can be implemented as a neural network that can include any appropriate types of neural network layers (e.g., fully connected layers, attention layers, convolutional layers, and so forth) in any appropriate number (e.g., 5 layers, or 10 layers, or 100 layers) and connected in any appropriate configuration (e.g., as a directed graph of layers).

A few examples of possible predictions 122 are described next.

In some implementations, the environment is a medical environment that includes a patient, and the prediction defines a predicted medical treatment to be applied to the patient. For instance, the prediction can include a respective score for each medical treatment in a set of medical treatments, where the score for a medical treatment defines a likelihood that the medical treatment should be applied to the patient. The set of medical treatments can include medical treatments corresponding to administering a drug to the patient, performing an intervention (e.g., surgery) on the patient, etc.

In some implementations, the environment is a medical environment that includes a patient, and the prediction defines a predicted medical diagnosis for the patient. For instance, the prediction can include a respective score for each medical diagnosis in a set of medical diagnoses, where the score for a medical diagnosis defines a likelihood that the medical diagnosis applies to the patient. The set of medical diagnoses can include, e.g., diagnoses for one more diseases, e.g., cancer, diabetes, heart failure, Alzheimer's, flu, measles, strep throat, sepsis, etc.

In some implementations, the environment is an agriculture environment (e.g., an environment where crops are cultivated) or an aquaculture environment (e.g., an environment where aquatic organisms are cultivated), and the prediction defines a predicted yield (e.g., measured in tons of crops or aquatic organisms), or a predicted amount of time until crops or aquatic organisms in the environment should be harvested (e.g., measured in days).

In some implementations, the environment is an industrial environment, and the prediction defines a predicted production level of the industrial environment over a predefined time range (e.g., 1 hour, 1 day, or 1 week), e.g., a number of units of product generated by a manufacturing facility, or a quantity of chemicals generated by a chemical processing facility, or a number of computing tasks completed by a data center facility, or an amount of energy produced by an energy production facility.

In some implementations, the environment is a scientific environment, and the prediction defines a predicted result of a scientific study, e.g., the health of subjects of the scientific study, e.g., the integrity of cell walls of a collection of cells at the conclusion of the study, or the weight of animals in a population of animals at the conclusion of the study.

In these example environments and many other real-world environments, capturing data corresponding to a modality oftentimes incur significant cost, e.g., in terms of resource consumption (e.g., consumption of energy or time), or in terms of risk (e.g., medical risk, e.g., resulting from exposing a patient to radiation from acquiring medical images of the patient, e.g., x-ray images or CT images). Moreover, processing data corresponding to certain modalities can also incur significant cost, e.g., in terms of computational resources (e.g., memory and computing power), e.g., for high-dimensional data such as image data, video data, or audio data.

Therefore, although the neural network system 100 could potentially receive input data corresponding to each and every modality in the set of two or more available modalities at each time step, the system may not actually do so, and may instead only acquire (and thereafter receive) input data corresponding to each modality in a proper subset of the set of two or more available modalities at each of one or more time steps. A proper subset includes at least one modality in the set of two or more available modalities, but less than all of the modalities in the set.

In the example of FIG. 1, the neural network system 100 could potentially receive data corresponding respectively to a set of three data modalities that might be made available to the system: data 102A corresponding to modality A, data 102B corresponding to modality B, and data 102C corresponding to modality C.

However, as illustrated, the actually acquired input data 102 is multi-modal data that only includes data corresponding respectively to two of the three available data modalities: data 102A corresponding to modality A, and data 102B corresponding to modality B. That is, data 102C corresponding to modality C is not acquired and is therefore not received by the system, and the actually acquired input data 102 does not include data 102C corresponding to modality C.

In other examples, the input data can potentially (but not necessarily) include data corresponding to a smaller (e.g., two) or larger (e.g., ten, one hundred, or more) set of available modalities. Analogously, in those examples, the actually acquired input data 102 can include data corresponding to each modality in a proper subset of the smaller or larger set of available modalities.

In particular, for each time step after a first time step in the sequence of time steps, the neural network system 100 uses the selection neural network 110 to make acquisition decisions that define, for each modality in the set of modalities, whether data corresponding to the modality will be selected for acquisition.

At any given time step after the first time step in the sequence of multiple time steps, the selection neural network 110 processes a network input that includes (i) previous observations 112 obtained for one or more preceding time steps and, optionally, (ii) data identifying the acquisition decision for any modality at any preceding time step, to generate a plurality of acquisition decisions for the given time step. Each acquisition decision corresponds to a respective modality from the set of multiple modalities and defines whether data corresponding to the modality should be selected for acquisition at the given time step. As will be explained further below, an “observation” refers to data that is generated by the data acquisition engine 130 from the acquired input data 102 and that is provided to the selection neural network 110 and/or prediction model 120 for further processing.

The selection neural network 110 can have any appropriate neural network architecture that allows the selection neural network 110 to generate acquisition decisions from previous observations. In particular, the selection neural network can include any appropriate types of neural network layers (e.g., fully connected layers, attention layers, convolutional layers, and so forth) in any appropriate number (e.g., 5 layers, or 10 layers, or 100 layers) and connected in any appropriate configuration (e.g., as a directed graph of layers).

As a particular example, the selection neural network 110 and the prediction model 120 can each be configured as a respective neural network having one of the architectures described in Andrew Jaegle, et al. PerceiverIO: A general architecture for structured inputs & outputs. In International Conference on Representation Learning, 2022.

In the example of FIG. 1, at a particular time step, the selection neural network 110 generates a total of three acquisition decisions: decision A corresponding to modality A, decision B corresponding to modality B, and decision C corresponding to modality C. Specifically, decision A defines that data corresponding to modality A should be selected for acquisition at the time step, decision B defines that data corresponding to modality B should be selected for acquisition at the time step, and decision C defines that data corresponding to modality C should not be selected for acquisition at the time step. The selection neural network 110 can generate different acquisition decisions at other time steps.

Each acquisition decision can be generated deterministically, e.g. by an output of the selection neural network 110. For example, an output layer of the selection neural network 110 can include a respective neuron corresponding to each modality; and each modality is selected for acquisition only if the activation of the neuron exceeds a predefined threshold. Alternatively, each acquisition decision can be generated stochastically e.g. where the output of the selection neural network 110 parameterizes a distribution from which the acquisition decision is sampled. For example, each acquisition decision can be a binary decision, with 0 indicating that data corresponding to a particular modality should not be selected for acquisition, and 1 indicating that data corresponding to the particular modality should be selected for acquisition.

The neural network system 100 then uses the data acquisition engine 130 to effectuate the acquisition decisions generated by the selection neural network 110. That is, the neural network system 100 provides the acquisition decisions to the data acquisition engine 130—and the data acquisition engine 130 causes data to be acquired only for modalities selected for acquisition at the given time step.

In the example of FIG. 1, the data acquisition engine 130 acquires, in accordance with the acquisition decisions generated by the selection neural network 110, data corresponding to modality A and data corresponding to modality B. The data acquisition engine 130 refrains from acquiring data corresponding to modality C.

In some implementations, the data acquisition engine 130 can effectuate (e.g. execute or enact) the acquisition decisions by passing an electronic signal to a sensor, or another electronic device having environment sensing capabilities that is communicatively coupled to the system, to capture data corresponding to one of the selected modalities. In response to receiving the electronic signal, the sensor operates to capture the data with or about the environment.

In some implementations, the data acquisition engine 130 can effectuate the acquisition decisions by generating and outputting a prompt for presentation to a user through a user interface device. The user interface device can be any appropriate stationary or mobile computing device, such as a desktop computer, a workstation in a medical environment, a tablet, a smartphone, or a smartwatch.

The prompt can help guide a user in capturing data according to the acquisition decisions generated by the selection neural network 110. The prompt can instruct the user on what modalities of data to capture. For example, the prompt can be presented within a window with text asking that data corresponding to one of the selected modalities should be captured. A user can interact with the user interface device to view the selected modality, and upload data corresponding to the selected modality after the data is captured.

The neural network system 100 then generates an observation 112 for the given time step from the input data 102, which is acquired by the data acquisition engine 130 in accordance with the plurality of acquisition decisions generated by the selection neural network 110.

The observation 112 for the given time step: (i) includes data corresponding to modalities, from the set of modalities, that are selected for acquisition at the given time step, and that (ii) excludes, i.e., does not include, data corresponding to modalities, from the set of modalities, that are not selected for acquisition at the given time step. After being generated, the observation 112 is then provided to the prediction model 120 for further processing.

In this way, although the data that is potentially available to the system includes multi-modal data corresponding respectively to the set of modalities, only data corresponding to a proper subset of the modalities in the set of modalities may actually be selected for acquisition by the neural network system 100. For example, only data that corresponds respectively to a small number of modalities within a relatively large number of modalities may be selected for acquisition by the neural network system 100, and, thereafter, used by the prediction model 120 to generate the prediction 122.

By incorporating the selection neural network 110 and acquiring data in accordance with the acquisition decisions generated by the selection neural network 110, the neural network system 100 can reduce the amount of computational resources consumed by the prediction process because repeatedly acquiring and subsequently processing data from all of the set of modalities is no longer necessarily required. Instead, at each of at least some of the time steps, only data from a relatively small number of selected modalities needs to be acquired and then processed.

The training engine 140, when included, can train the selection neural network 110 and, optionally, the prediction model 120 to determine trained parameter values of the selection neural network 110 and, optionally, trained parameter values of the prediction model 120 that enable the selection neural network 110 to generate acquisition decisions that can result in reduced consumption of computational resources by the system while still maintaining predictive performance, e.g., in terms of the accuracy of the predictions 122. Thus, in some implementations, the selection neural network 110 and the prediction model 120 may be jointly trained by the training engine 140.

In the example of FIG. 1, the training engine 140 includes or has access to a cost computation engine 145. The cost computation engine 145 is configured to compute acquisition costs associated with the modalities that are selected for acquisition according to the acquisition decisions generated by the selection neural network 110.

The training engine 140 can thus apply a reinforcement learning technique that uses a reward derived from the acquisition costs to train the selection neural network 110 jointly with the prediction model 120 to optimize a trade-off between acquisition cost and predictive performance.

In particular, the training engine 140 can train the selection neural network 110 and the prediction model 120 to achieve an acceptable predictive performance while minimizing acquisition cost across the available modalities, thus enabling more efficient use of resources (e.g., energy resources or computational resources) and reduction of risk (e.g., medical risk).

The cost computation engine 145 can be configured to compute the acquisition cost for a modality in a set of modalities based on any appropriate criteria. A few examples of possible criteria for setting acquisition costs for modalities are described next.

In some implementations, the acquisition cost for a modality can be based at least in part on an amount of resource usage (e.g., energy or time) required to acquire data corresponding to the modality.

In some implementations, the acquisition cost for a modality can be based at least in part on an amount of risk required to acquire data corresponding to the modality. For example, in a medical environment, acquiring data corresponding to a biopsy modality may incur a risk of infection in the patient, and acquiring data corresponding to an x-ray modality may incur a risk of exposing the patient to unhealthy levels of radiation. An amount of risk may be determined based on statistics characterizing different outcomes (e.g. patient outcomes) when data corresponding to the modality is acquired.

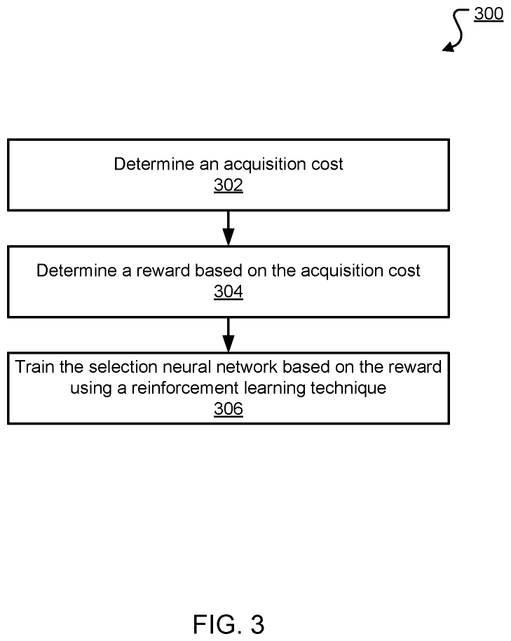

In some implementations, the acquisition cost for a modality can be based at least in part on a level of disruption caused by acquiring data corresponding to the modality. For example, in an industrial environment, acquiring data corresponding to a modality can include running diagnostic tests that reduce production of the industrial facility. As another example, in a scientific environment, acquiring data corresponding to a modality can include disrupting conditions in the environment (e.g., by performing tests on one or more subjects in the environment) in a manner that could compromise the validity or accuracy of results of the experiment. Training the selection neural network 110 will be described further below with reference to FIGS. 3-6.

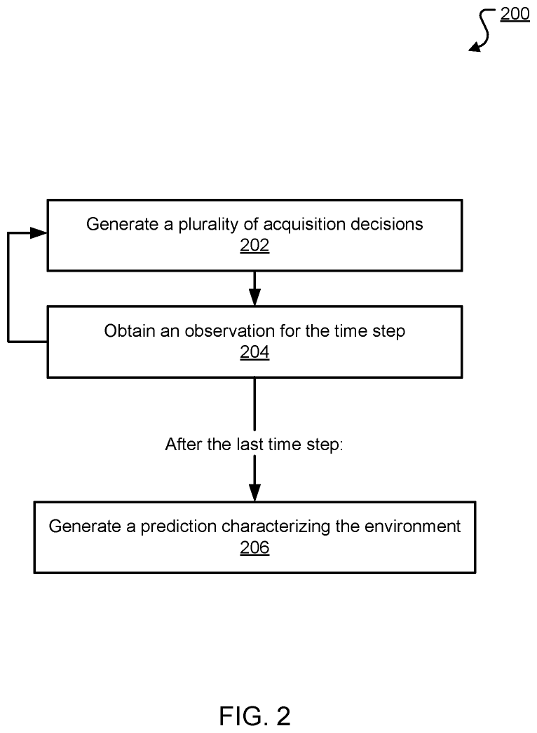

FIG. 2 is a flow diagram of an example process 200 for generating a prediction characterizing an environment. For convenience, the process 200 will be described as being performed by a system of one or more computers located in one or more locations. For example, a neural network system, e.g., the neural network system 100 of FIG. 1, appropriately programmed, can perform the process 200.

The environment can be any appropriate environment, e.g., a real-world environment, e.g., a medical environment, an agriculture environment, an aquaculture environment, an industrial environment, or a scientific environment.

The system repeatedly performs steps 202 and 204 to obtain a respective observation characterizing a state of an environment for each time step in a sequence of multiple time steps. That is, the system performs one iteration of steps 202 and 204 for each time step in the sequence of multiple time steps.

In some implementations, the number of time steps is fixed (predefined). For example, the system can generate a sequence-level prediction after a predefined number of time steps have elapsed. In other implementations, the number of time steps is flexible, and different sequences can include varying numbers of time steps. For example, the system can repeatedly perform iterations of steps 202 and 204 until a termination signal (e.g., a flag or another indicator) is received at a given time step indicating that the given time step is the last time step in the sequence. For example, a flag can be set to a first value if the given time step is not the last time step in a sequence and the flag can be set to a second value if the given time step is the last time step in the sequence. The termination signal may be based on the prediction(s) generated by the system.

For each time step after a first time step in the sequence of multiple time steps, the system processes, using a selection neural network, a network input that includes (i) observations obtained for one or more preceding time steps and, optionally (ii) data identifying the acquisition decision for any modality at any preceding time step, to generate a plurality of acquisition decisions for the time step (step 202). An “observation” refers to data that is generated by the data acquisition engine 130 from the acquired input data 102 and that is provided to the selection neural network 110 and/or prediction model 120 for further processing. Each acquisition decision corresponds to a respective modality from a set of multiple modalities, and defines whether data corresponding to the modality is selected for acquisition at the time step.

For the first time step, because there is no preceding time step, some implementations of the system can instead provide a predetermined network input, i.e., an input having predetermined values, for processing by the selection neural network. Some other implementations of the system can alternatively acquire a default (e.g., random or predefined) set of modalities, i.e., without using the selection neural network to generate any acquisition decisions for the first time step.

The system obtains an observation for the time step in accordance with the plurality of acquisition decisions generated by the selection neural network (step 204). For example, the system can use a data acquisition engine to acquire data corresponding to each modality that is selected for acquisition according to the acquisition decisions, and then include the acquired data in the observation.

In particular, the observation (i) includes data corresponding to modalities, from the set of modalities, that are selected for acquisition at the time step, and (ii) does not include data corresponding to modalities, from the set of modalities, that are not selected for acquisition at the time step.

The selected modalities may, and generally will, vary from one time step to another. In other words, the system may obtain data corresponding to different modalities at different time steps.

In some examples, for one or more time steps in the sequence of multiple time steps, the system can obtain an observation that includes data corresponding to all of the modalities in the set. In another example, for one or more time steps, the system can obtain an observation that includes data corresponding to a proper subset of the set of modalities (and does not include data corresponding to any remaining modality that is not in the proper subset). A “proper” subset of a set is a subset that includes one or more but not all of the elements in the set. In another example, for one or more time steps, the system can obtain a null observation that does not include data corresponding to any modality in the set.

After having performed the iteration of steps 202 and 204 for the last time step in the sequence of multiple time steps, the system processes, using a prediction model, a model input that includes the observation for each time step in the sequence of time steps to generate a prediction characterizing the environment (step 206).

An example algorithm for generating a prediction is shown below.

Algorithm 1 A2MT

| Inputs: Test input x, agent π, model f. | |
|---|---|
| 1: | for t = 1 to T do |
| 2: | Sample αₜ ~ π(·\|\tilde{x}_{1:t-1}, α_{1:t-1}; θ). |
| 3: | for m = 1 to M do |
| 4: | if a_{t, m} = 1 then |
| 5: | Acquire: \tilde{x}_{t, m} ← x_{t, m}. |
| 6: | else |
| 7: | Do not acquire: \tilde{x}_{t, m} ← ∅. |
| 8: | end if |
| 9: | end for |
| 10: | end for |
| 11: | Return prediction f(\tilde{x}_{1:T}). |

In Algorithm 1, each input x includes a sequence of observations xᵢ=(x_{i,1}, . . . , x_{i,T}). At each time step t, the observation x_{i,t} includes data corresponding to M modalities x_{i,t}=(x_{i,t,1}, . . . , x_{i,t,M}). Each modality may be high-dimensional, x_{i,t,m}∈R^{d}^{ₘ}. For example, x_{i,t,m} could be a single frame in a video having dimensionality dₘ=H·W·C, where H is height, W is width, and C is number of color channels.

At each timestep t∈(1, . . . , T), a plurality of acquisition decisions across modalities aₜ=(a_{t,1}, . . . , a_{t,M}) are generated by sampling from the output of the selection neural network (which is referred to in Algorithm 1 as an agent): aₜ˜π(·|\tilde{x}_{1:t-1}, a_{1:t-1}; θ). Here, a_{t,M}∈(0,1) is a binary indicator of whether modality m was acquired at time step t. \tilde{x} is used instead of x to highlight that the input data may contain missing entries, and θ are the parameters of the selection neural network. At each timestep t, and for each modality m, data x_{m,t} corresponding to the modality m is acquired only if a_{m,t}=1.

By repeatedly performing the process 200, the system can generate different predictions that characterize the same or different aspects of the environment. That is, the process 200 can be performed as part of generating a prediction from a sequence of observations for which the desired output, i.e., the desired prediction that should be generated by the system from the sequence of observations, is not known. One or more actions may be performed based on the prediction(s). For example, an agent, such as an electromechanical agent, interacting with a real-world environment to perform a task, may select one or more action to perform in the real-world environment according to the prediction(s).

Some of all of the steps of the process 200 can also be performed as part of processing sequences of observations derived from a set of training data, i.e., sequences of observations derived from input data for which the predictions that should be generated by the system are known, in order to train the trainable components of the system to determine trained values for the parameters of these components.

FIG. 3 is a flow diagram of an example process 300 for training a selection neural network. For convenience, the process 300 will be described as being performed by a system of one or more computers located in one or more locations. For example, a neural network system, e.g., the neural network system 100 of FIG. 1, appropriately programmed, can perform the process 300.

During training, process 300 can be performed subsequent to process 200 on each training input selected from a set of training data derived from a plurality of temporal sequences of input data generated within or about an environment (e.g., one of the physical environments mentioned above or a computer simulation of one of these physical environments). That is, for each training input, the system performs process 200 to generate a prediction characterizing the environment using the selection neural network and in accordance with the current values of the parameters of the selection neural network, and then performs process 300 to determine one or more updates to the parameter values of the selection neural network based on the prediction generated in process 200.

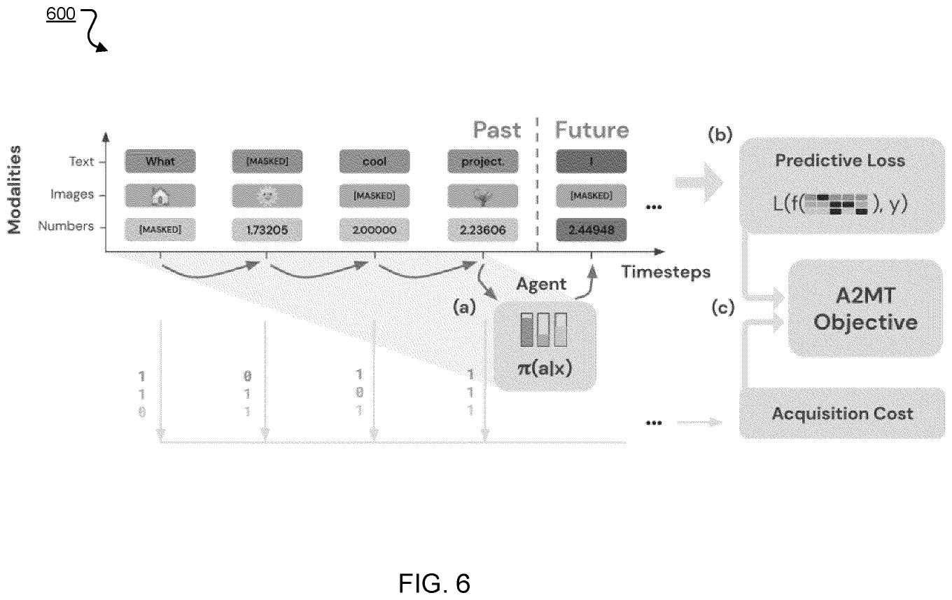

The process 300 is illustrated in FIG. 6, which shows an example of generating a prediction using the selection neural network and determining one or more updates to the parameter values of the selection neural network based on the prediction.

As illustrated in FIG. 6, at any given time step, the selection neural network (“Agent”) generates a total of three acquisition decisions: a first decision corresponding to a text modality, a second decision corresponding to an image modality, and a third decision corresponding to a numeric modality. To generate these acquisition decisions for the given time step, the selection neural network processes a network input that includes observations obtained for one or more preceding time steps to generate an output π which parameterizes a distribution from which the acquisition decision is sampled.

For example, at a first time step, the first decision defines that text data should be selected for acquisition at the time step, the second decision defines that image data should be selected for acquisition at the time step, and the third decision defines that numeric data should not be selected for acquisition at the time step. As represented by the [masked] tokens, some input data may contain missing entries.

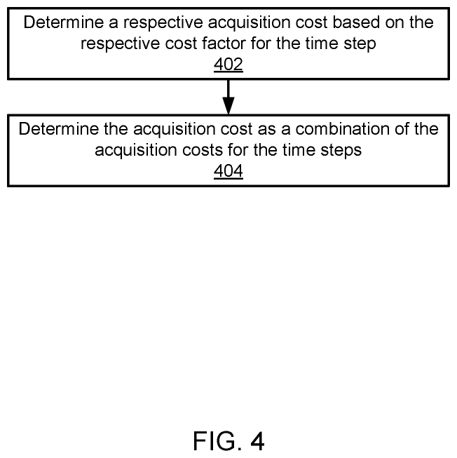

After generating the prediction based on the modalities selected for acquisition at each time step by the selection neural network, the system determines an acquisition cost based on the respective modalities selected for acquisition at each time step in the sequence of time steps (step 302). In some implementations, each modality in the set of modalities is associated with a respective cost factor. In these implementations, the system can perform sub-steps 402-404, as is explained in more detail with reference to FIG. 4, to determine the acquisition cost.

FIG. 4 is a flow diagram of sub-steps 402-404 of step 302 of the process of FIG. 3.

In implementations where each modality in the set of modalities is associated with a respective cost factor, the system can determine, for each time step in the sequence of time steps, a respective acquisition cost for the time step based on the respective cost factor associated with each modality selected for acquisition at the time step (step 402). For example, the acquisition cost for the time step can be computed as a sum, either weighted or unweighted, of the cost factor associated with each modality selected for acquisition at the time step.

The system determines the acquisition cost as a combination of the acquisition costs for the time steps in the sequence (step 404). For example, the acquisition costs can be computed as a sum, either weighted or unweighted, over the respective acquisition costs for the time steps in the sequence.

For each of one or more of the modalities, the cost factor for the modality is based at least in part on an amount of resource usage required to capture data corresponding to the modality. For example, the resource usage required to capture data corresponding to the modality characterizes at least energy usage required to capture data corresponding to the modality. As another example, the resource usage required to capture data corresponding to the modality characterizes at least an amount of time required to capture data corresponding to the modality.

Additionally or alternatively, for each of one or more of the modalities, the cost factor for the modality is based at least in part on a risk associated with capturing data corresponding to the modality. When the environment is a medical environment that includes a patient, for example, the risk associated with capturing data corresponding to the modality is based at least in part on a medical risk to the patient resulting from capturing data corresponding to the modality.

The system determines a reward based at least in part on the acquisition cost the selected modalities (step 304). The acquisition cost can be included in the reward, which is typically a numeric value, in any appropriate manner.

For example, the system can determine the reward based at least in part on a comparison of the acquisition cost to a threshold referred to as a “cost budget.” In some implementations, the system can reduce the reward by a predefined or adaptive amount if the acquisition cost exceeds the cost budget. The cost budget can indicate, e.g., an acceptable level of acquisition cost, e.g., an acceptable amount of energy usage, or an acceptable amount of medical risk (e.g., based on a tolerable amount of radiation exposure for a patient), or an acceptable amount of computational resources (e.g., memory and computing power) used for processing data from the acquired modalities.

In some implementations, the reward depends on both (i) the acquisition cost and (ii) a prediction error that measures an error in the prediction generated by the prediction model. In these implementations, as illustrated in FIG. 6, the reward can for example be computed as an expectation value:

$$R = 𝔼 [ - C ( a ) - ℒ ( f ( {\tilde{x}}_{1 : T} ) , y ) ] , where C ( a ) = {\sum }_{t = 1}^{T} {\sum }_{m = 1}^{M} {c}_{m} {a}_{t , m} . \qquad ( 1 )$$

Here, the expectation is over a training input (x,y), where x is a sequence of observations and y is the ground truth prediction, a represents acquisition decisions generated by the selection neural network; C(a) represents the total acquisition cost of the sequence of observations; Cₘ is a modality-specific cost factor, and [symbol] (f(\tilde{x}_{1:T}), y) is log likelihood loss of the prediction generated by the prediction model with respect to the ground truth prediction (although other loss functions may of course be used, i.e. loss functions comparing the prediction generated by the prediction model with the ground truth prediction).

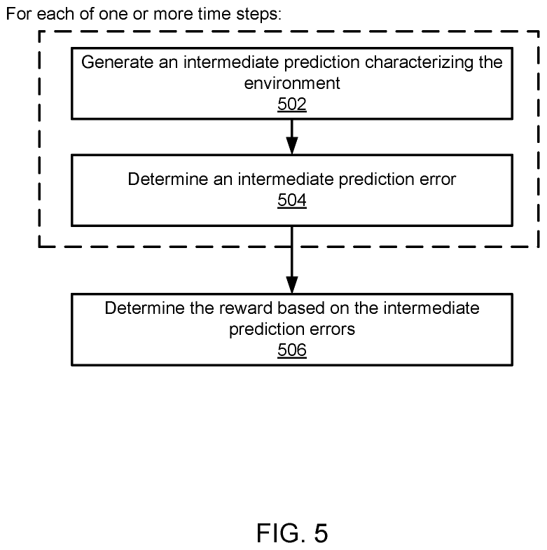

Optionally, in some implementations, the system adds intermediate prediction errors to the reward, e.g., the reward computed using Equation (1). The intermediate prediction errors, when used, encourage the selection neural network to decrease the prediction error. In these implementations, the system can perform sub-steps 502-506, as is explained in more detail with reference to FIG. 5, to determine the reward.

FIG. 5 is a flow diagram of sub-steps 502-506 of step 304 of the process of FIG. 3.

For each of one or more time steps in the sequence of time steps, the system processes a model input that includes the observation for the time step and observations for one or more preceding time steps in the sequence of time steps using the prediction model to generate an intermediate prediction characterizing the environment (step 502).

For each of the one or more time steps in the sequence of time steps, the system determines an intermediate prediction error that measures an error in the intermediate prediction generated by the prediction model (step 504).

The system determines the reward based at least in part on the intermediate prediction errors that have been determined for the one or more time steps (step 506). For example, the system can add the intermediate prediction errors to the reward computed using Equation (1). The intermediate prediction errors can for example be computed as:

$$I = - α {\sum }_{t = 1}^{T} ( ℒ ( f ( {\tilde{x}}_{1 : t} ) , y ) - γℒ ( f ( {\tilde{x}}_{1 : t - 1} ) , y ) ) , \qquad ( 2 )$$

where α is a hyperparameter (e.g., a predefined constant value), γ is the discount factor, x is a sequence of observations and y is the ground truth prediction, and [symbol] (f(\tilde{x}_{1:T}), y) is log likelihood loss of the prediction generated by the prediction model with respect to the ground truth prediction.

The system trains the selection neural network based on the reward using a reinforcement learning technique to adjust the values of the parameters of the selection neural network (step 304). In particular, the system trains the selection neural network to generate acquisition decisions that maximize the reward that is determined based at least in part on the acquisition cost. For example, the reinforcement learning technique can be a policy gradient technique, e.g., an advantage actor critic (A2C) policy gradient technique, that applies Gumbel parameterization to the (discrete) acquisition decisions.

In some implementations, the system also trains the prediction model based on the reward, e.g., the reward computed using Equation (1), which depends on both the acquisition cost and the prediction error, to simultaneously adjust the values of the parameters of the prediction model.

For example, the system can train the prediction model and the selection neural network together to jointly update the parameter values of both the selection neural network and the prediction model, e.g., in order to allow the prediction model to adapt specifically to the combinations of modalities frequently selected by the selection neural network. In this example, as the parameter values of the prediction model are updated, the rewards received by the selection neural network may consequently change.

Alternatively, in other implementations, the system can train the prediction model separately from the training of the selection neural network (during which the parameter values of the prediction model are held fixed), e.g., based on optimizing an objective function that depends on the prediction error of the prediction machine learning model.

For example, the system can pre-train the prediction model to process masked sequences of observations to generate corresponding predictions. The system then trains the selection neural network to update the parameter values of the selection neural network, while holding the pre-trained parameter values of the prediction model fixed.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework or a JAX framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A method performed by one or more computers, the method comprising:
obtaining a respective observation characterizing a state of an environment for each time step in a sequence of multiple time steps, comprising, for each time step after a first time step in the sequence of time steps:
processing a network input that comprises observations obtained for one or more preceding time steps using a selection neural network to generate a plurality of acquisition decisions,
wherein each acquisition decision corresponds to a respective modality from a set of multiple modalities and defines whether data corresponding to the modality is selected for acquisition at the time step;
obtaining an observation for the time step, wherein the observation: (i) includes data corresponding to modalities, from the set of modalities, that are selected for acquisition at the time step, (ii) does not include data corresponding to modalities, from the set of modalities, that are not selected for acquisition at the time step; and
processing a model input that includes the observation for each time step in the sequence of time steps using a prediction model to generate a prediction characterizing the environment.

2. The method of claim 1, further comprising:
determining an acquisition cost based on the respective modalities selected for acquisition at each time step in the sequence of time steps;
determining a reward based at least in part on the acquisition cost; and
training the selection neural network based on the reward using a reinforcement learning technique.

3. The method of claim 2, wherein each modality in the set of modalities is associated with a respective cost factor, and wherein determining the acquisition cost comprises:
determining, for each time step in the sequence of time steps, a respective acquisition cost for the time step based on the respective cost factor associated with each modality selected for acquisition at the time step; and
determining the acquisition cost as a combination of the acquisition costs for the time steps.

4. The method of claim 3, wherein for each time step in the sequence of time steps, determining the acquisition cost for the time step comprises:
determining the acquisition cost for the time step as a sum of the cost factor associated with each modality selected for acquisition at the time step.

5. The method of claim 3, wherein determining the acquisition cost as a combination of the acquisition costs for the time steps comprises:
determining the acquisition costs as a sum over the acquisition costs for the time steps.

6. The method of claim 3, wherein for one or more of the modalities, the cost factor for the modality is based at least in part on an amount of resource usage required to capture data corresponding to the modality.

7. The method of claim 6, wherein the resource usage required to capture data corresponding to the modality characterizes at least energy usage required to capture data corresponding to the modality.

8. The method of claim 6, wherein the resource usage required to capture data corresponding to the modality characterizes at least an amount of time required to capture data corresponding to the modality.

9. The method of claim 3, wherein for one or more of the modalities, the cost factor for the modality is based at least in part on a risk associated with capturing data corresponding to the modality.

10. The method of claim 9, wherein the environment comprises a patient, and the risk associated with capturing data corresponding to the modality is based at least in part on a medical risk to the patient resulting from capturing data corresponding to the modality.

11. The method of claim 2, further comprising:
determining a prediction error that measures an error in the prediction generated by the prediction model; and
determining the reward based on both: (i) the acquisition cost, and (ii) the prediction error.

12. The method of claim 1, wherein the prediction model is a machine learning model.

13. The method of claim 12, wherein the prediction model comprises a neural network.

14. The method of any claim 12, further comprising:
training the prediction machine learning model to optimize an objective function that depends on a prediction error of the prediction machine learning model.

15. The method of claim 1, wherein for each time step in the sequence of time steps, the network input to the selection neural network at the time step further comprises:
data identifying the acquisition decision for any modality at any preceding time step.

16. The method of claim 2, further comprising:
for each of one or more time steps in the sequence of time steps:
processing a model input that includes the observation for the time step and observations for one or more preceding time steps in the sequence of time steps using the prediction model to generate an intermediate prediction characterizing the environment; and
determining an intermediate prediction error that measures an error in the intermediate prediction generated by the prediction model; and
determining the reward based at least in part on the intermediate prediction errors.

17. The method of claim 1, wherein the set of modalities includes an imaging modality, and wherein data corresponding to the imaging modality comprises image data.

18. The method of claim 17, wherein the set of modalities includes a medical imaging modality.

19. (canceled)

20. (canceled)

21. (canceled)

22. (canceled)

23. (canceled)

24. (canceled)

25. A system comprising:
one or more computers; and
one or more storage devices communicatively coupled to the one or more computers, wherein the one or more storage devices store instructions that, when executed by the one or more computers, cause the one or more computers to perform operations comprising:
obtaining a respective observation characterizing a state of an environment for each time step in a sequence of multiple time steps, comprising, for each time step after a first time step in the sequence of time steps:
processing a network input that comprises observations obtained for one or more preceding time steps using a selection neural network to generate a plurality of acquisition decisions,
wherein each acquisition decision corresponds to a respective modality from a set of multiple modalities and defines whether data corresponding to the modality is selected for acquisition at the time step;
obtaining an observation for the time step, wherein the observation: (i) includes data corresponding to modalities, from the set of modalities, that are selected for acquisition at the time step, (ii) does not include data corresponding to modalities, from the set of modalities, that are not selected for acquisition at the time step; and
processing a model input that includes the observation for each time step in the sequence of time steps using a prediction model to generate a prediction characterizing the environment.

26. One or more non-transitory computer storage media storing instructions that when executed by one or more computers cause the one or more computers to perform operations comprising:
obtaining a respective observation characterizing a state of an environment for each time step in a sequence of multiple time steps, comprising, for each time step after a first time step in the sequence of time steps:
processing a network input that comprises observations obtained for one or more preceding time steps using a selection neural network to generate a plurality of acquisition decisions,
wherein each acquisition decision corresponds to a respective modality from a set of multiple modalities and defines whether data corresponding to the modality is selected for acquisition at the time step;
obtaining an observation for the time step, wherein the observation: (i) includes data corresponding to modalities, from the set of modalities, that are selected for acquisition at the time step, (ii) does not include data corresponding to modalities, from the set of modalities, that are not selected for acquisition at the time step; and
processing a model input that includes the observation for each time step in the sequence of time steps using a prediction model to generate a prediction characterizing the environment.
